(19) United States
(12) Patent Application Publication
(10) Pub. No.: US 2002/0188336 A1
(43) Pub. Date: Dec. 12, 2002

(54) Method of enhancing and regularizing specific autonomic nervous and endocrine functions through the continuous and discriminate thermal or electric stimulation of specific discrete skin areas

(76) Inventors: Goetz Friedrich Bothe Loncar, Tepoztlan (MX); David Ignacio Garduno, Tepoztlan (MX)

Correspondence Address: DAVID I. GARDUNO, NETZAHUALCOYOTL 36, VALLE DE ATONGO, TEPOZTLAN, 62520, MX

(21) Appl. No.: 10/157,151

(22) Filed: May 30, 2002

Related U.S. Application Data
(60) Provisional application No. 60/296,487, filed on Jun. 8, 2001.

(51) Int. Cl. A61F 7/00; A61F 7/12

(52) U.S. Cl. 607/96

(57) ABSTRACT

In accordance with the present invention, a method for enhancing and regularizing specific autonomic nervous and endocrine functions is disclosed whereby specific discrete skin areas are stimulated with copper and aluminum plates. The discriminate stimulation of such specific discrete skin areas with copper and aluminum plates enhances, in turn, specific autonomic nervous and endocrine functions.

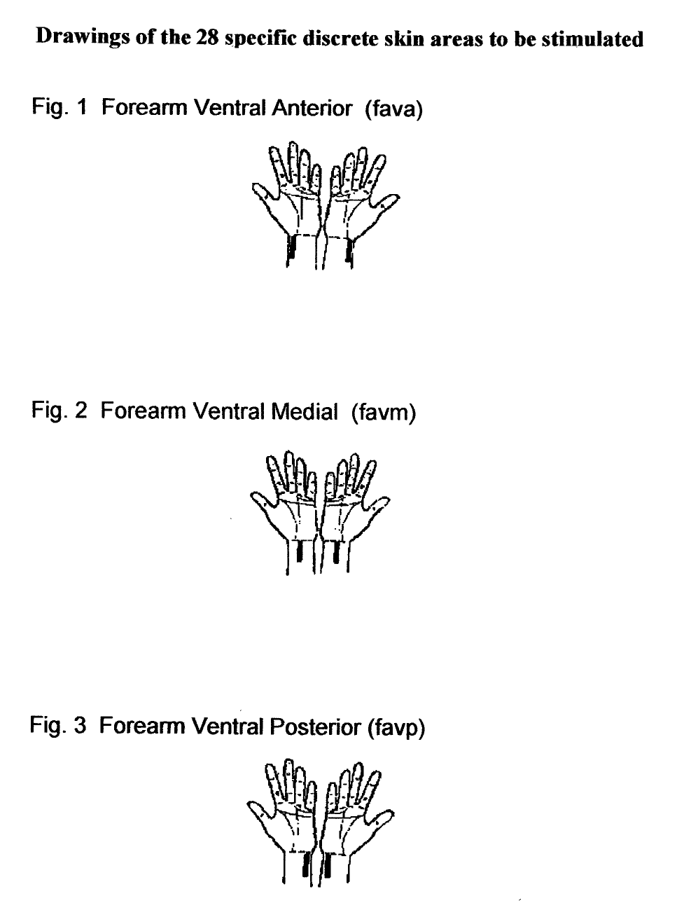

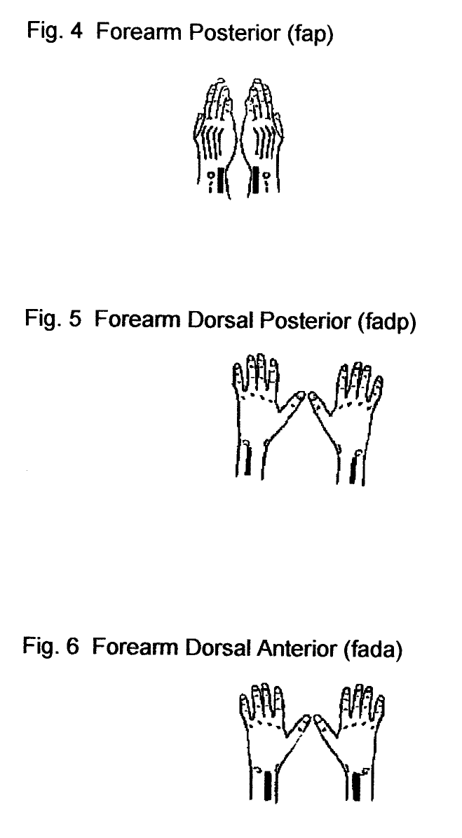

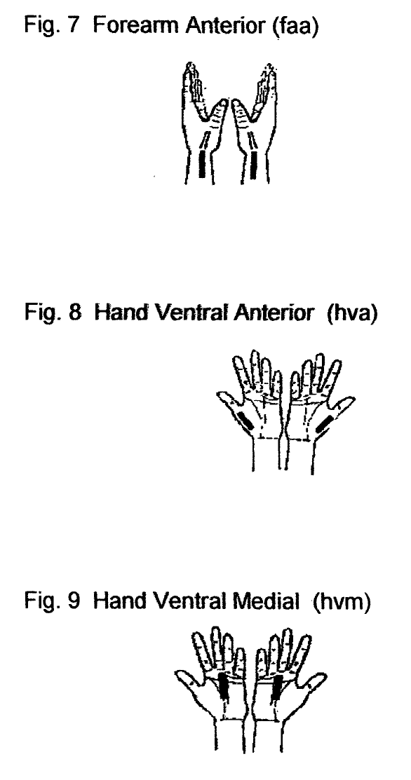

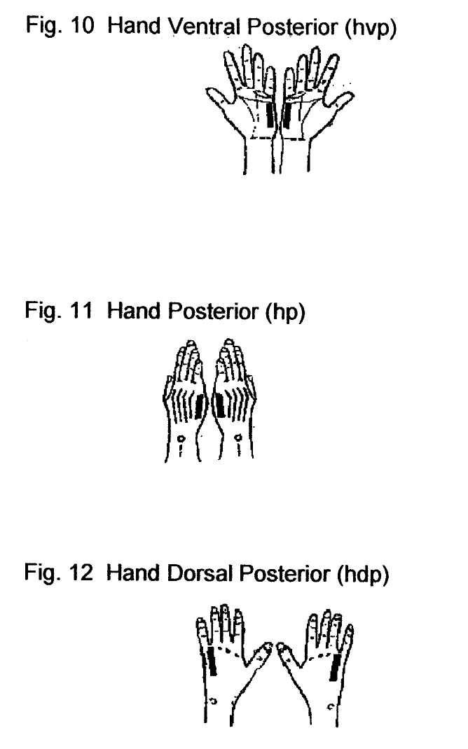

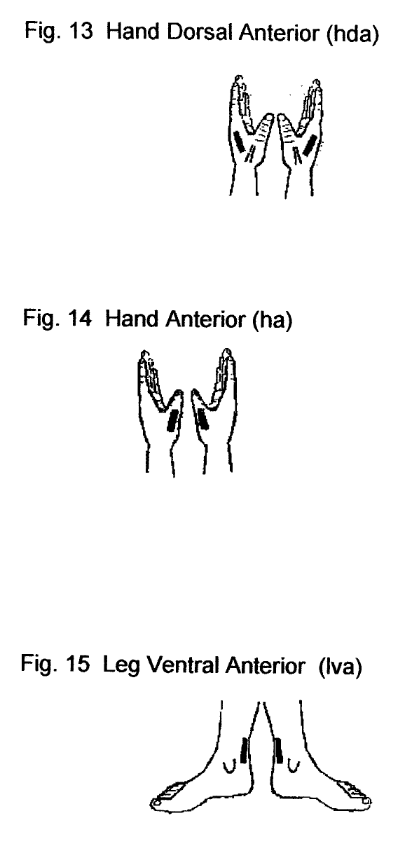

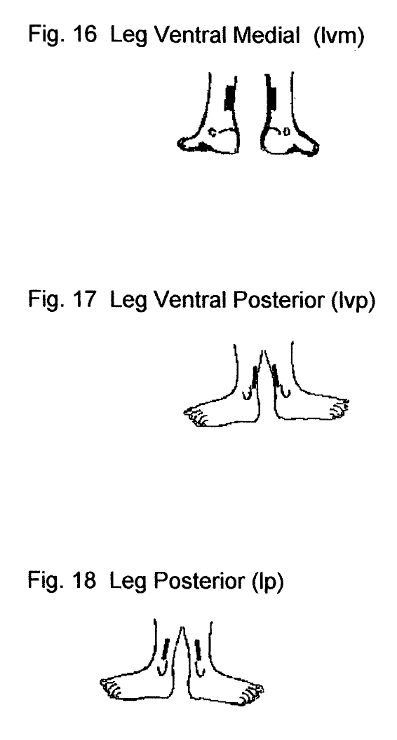

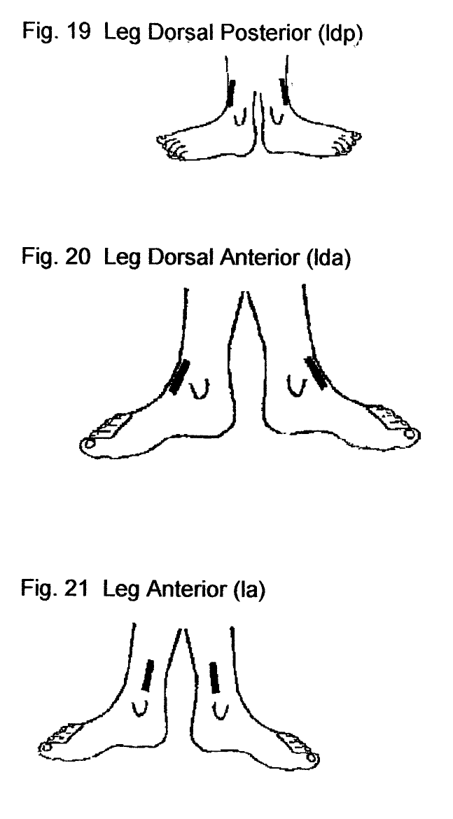

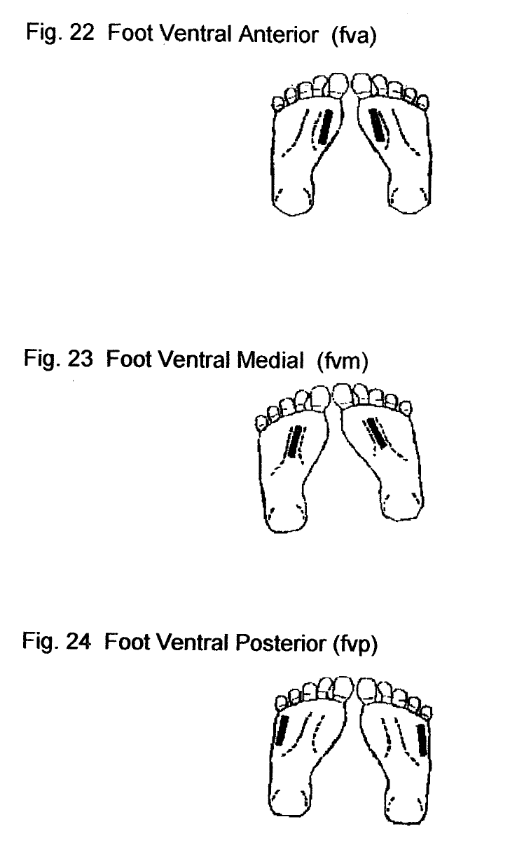

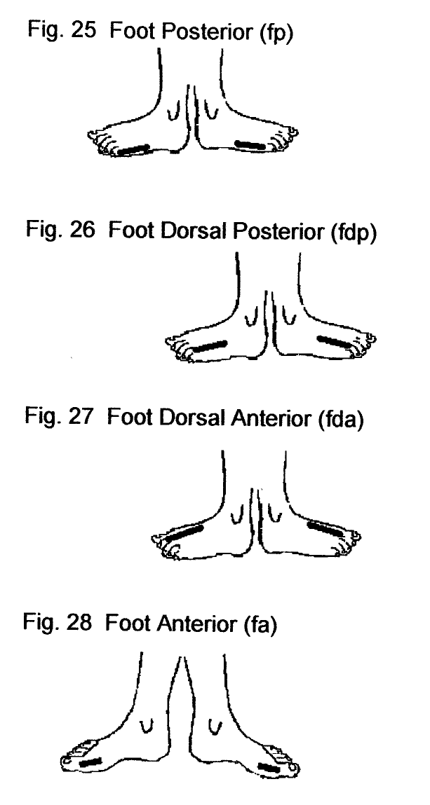

The present Non-Provisional Patent Application is related to a previous Provisional Patent Application with the following references:

APPLICATION NUMBER: 60/296,487; FILING DATE: Jun. 08, 2001

### CROSS-REFERENCE TO RELATED APPLICATIONS

Not Applicable

### STATEMENT REGARDING FEDERALLY SPONSORED RESEARCH OR DEVELOPMENT

Not Applicable

### REFERENCE TO SEQUENCE LISTING, A TABLE, OR A COMPUTER LISTING COMPACT DISK APENDIX

Not Applicable

### BACKGROUND OF THE INVENTION

The present invention is based on the discovery that the continuous and discriminate stimulation of specific discrete skin receptors with copper and aluminum plates enhances specific autonomic nervous and endocrine functions. The fields of research that constitute the background of the present invention are:

1. Research intended to elucidate the cytoarchitecture of neuronal circuits by which specific endocrine and autonomic regulatory activities are controlled. Particularly important is the research work concerning thermoregulation and its endocrine and autonomic effectors.

2. Research concerning the expression of Hox and other regulatory genes during limb pattern formation.

3. Research intended to elucidate the mechanism by which thermal and polymodal receptors are activated.

A list of references related to the present invention is provided:

1. Hemingway A: Shivering. Physiol Rev 43: 397-422, 1963.

2. Kleinebekel D and Klussmann F W: Shivering. In: Thermoregulation, Physiology and Biochemistry, ed. Schönbaum E and Lomax P, Pergamon Press, New York, Oxford, Beijing, Frankfurt, Sao Paulo, Sydney, Tokyo, and Toronto, pp 235-253, 1990.

3. Maskrey M: Metabolic and acid-base implications of thermal panting. In: Thermal Physiology, ed. Hales J R S, Raven Press, New York, pp 347-352, 1984.

4. Rothwell N J, Stock M J, and Stribling D: Diet-induced thermogenesis. In: Thermoregulation, Physiology and Biochemistry, ed. Schönbaum E and Lomax P, Pergamon Press, New York, Oxford, Beijing, Frankfurt, Sao Paulo, Sydney, Tokyo, and Toronto, pp 309-326, 1990.

5. Brüick K: Heat production and temperature regulation. In: Perinatal Physiology, ed. Stave U, Plenum Publ Corp, New York, pp 455-498, 1978.

6. Nicholls D G and Locke R M: Thermogenic mechanisms in brown fat. Physiol Rev 64: 1-64,1984.

7. Cannon B and Nedergaard J: Brown adipose tissue: molecular mechanisms controlling activity and thermogenesis. In: New Perspectives in Adipose Tissue: Structure, Function and Development, ed. Cryer A and Van R L R, Butterworths, London, pp 223-270,1985.

8. Nedergaard, J. and Cannon, B. The uncoupling protein thermogenin and mitochondrial thermogenesis. New Comprehens. Biochem 23:385-420, 1992.

9. Freake H C: Uncoupling proteins: beyond brown adipose tissue. Nutr Rev 56: 185-189, 1998.

10. Jansky, L. Humoral thermogenesis and its role in maintaining energy balance. Physiol. Rev. 75:237-259,1995.

11. Iriki M, Riedel W, and Simon E: Patterns of differentiation in various sympathetic efferents induced by changes in blood gas composition and by central thermal stimulation in anesthetized rabbits. Jpn. J. Physiol. 22:585-602, 1972.

12. Buijs R M and Kalsbeek A: Anatomy and physiology of vasopressin pathways: from temperature regulation to corticotrophin-inhibiting transmission. In: Neurohypophysis: Recent Progress of Vasopressin and Oxytocin Researsch, ed. Saito T, Kurokawa K, and Yoshida S, Elsevier, Amsterdam, Lausanne, New York, Oxford, Shannon, and Tokyo, pp 57-65, 1995.

13. Roth J: Immunological and neuroendocrine modulation of fever in stress. Progr Brain Res 115:177-192, 1998.

14. Roth J, Schulze K, Simon E, and Zeisberger E: Alteration of endotoxin fever and release of arginine vasopressin by dehydration in the guinea pig. Neuroendocrinology 56: 680-686,1992.

15. Riedel W, Städter W, and Gray D A: Activation of thyrotropin-releasing hormone (TRH) neurons by cold or after thyroidectomy inhibits antidiuretic hormone (ADH) secretion in febrile rabbits. J Auton Nerv Syst (Suppl): 543-553, 1986.

16. Clark W G and Fregly M J: Evidence for roles of brain peptides in thermoregulation. In: Handbook of Physiology, Section 4, Environmental Physiology, ed. Fregly M J, and Blatteis C M, Am Physiol Soc, Oxford Univ Press, New York, Vol. 1, pp 139-153, 1996.

17. Wilson K M and Fregly M J: Angiotensin H-induced hypothermica in rats. J Appl Physiol 58: 534-543, 1985.

18. Lin M T, Chandra A, and Jou J J: Angiotensin II inhibits both heat production and heat loss mechanisms in the rat. Can J Physiol Pharmacol 58: 909-914, 1980.

19. Huang B -S, Kluger M J, and Malvin R L: Thermal responses to central angiotensin II, SQ 20881, and dopamine infusions in sheep. Am J Physiol (Regul Integr Comp Physiol) 248: R371-R377, 1985.

20. Shido O and Nagasaka T: Effects of intraventricular antiotensin II on heat balance at various ambient temperatures in rats. Jpn J Physiol 35: 163-167, 1985.

21. Kregel K C, Strauss K, and Unger T: Modulation of autonomic nervous system adjustments to heat stress by central ANG II receptor antogonism. Am J Physiol (Regul Integr Comp Physiol) 266: R1985-R1991, 1994.

22. Horowitz M, Kaspler P, Simon E, and Gerstberger R: Heat acclimation and hypohydration: the involvement of central angiotensin II receptors in thermoregulation Am J Physiol (Regul Integr Comp Physiol) 277: R47-R55, 1999.

23. Keil R., Gerstberger R., and Simon E. Hypothalamic thermal stimulation modulates vasopressin release in hyperosmoically stimulated rabbits. Am. J. Physiol (Regul. Integr. Comp. Physiol) 267: R1089-R1097, 1994.

24. Merker G, Blähser S, and Zeisberger E. Reactivity pattern of vasopressin-containing neurons and its relation to the antipyretic reaction in the pregnant guinea pig. Cell Tissue Res 212: 47-61, 1980

25. Zeisberger E: The role of septal peptides in thermoregulation and fever. In: Thermoreception and Temperature Regulation, ed. Bligh J and Voigt K, Springer, Berlin, pp 273-283, 1990.

26. Landgraf R, Malkinson T J, Veale W L, Lederis K, and Pittman Q J: Vasopressin and oxytocin in rat brain in response to prostaglandin fever. Am J Physiol (Regul Intgr Comp Physiol) 259: R1056-R1062, 1990.

27. Kiyohara T, Miyata S, Nakamura T, Shido O, Nakashima T, and Shibata M: Differences in Fos expression in the rat brains between cold and warm ambient exposures Brain Res Bull 38: 193-201, 1995

28. Miyata S, Ishiyama M, Shido O, Nakashima T, and Shibata M: Central mechanism of neural activation with cold acclimation of rats using Fos immunocytochemistry Neurosci Res 22: 209-218, 1995.

29. Horowitz M and Meiri U: Thermoregulatory activity in the rat: effects of hypohydration, hypovolemia and hypertoncity and their interaction with short-term heat acclimation. Comp Biochem Physiol A 82: 577-582, 1985.

30. Patronas P, Horowitz M, Simon E, and Gerstberger R: Differential stimulation of c-fos expression in hypothalamic nuclei of the rat brain during short-term heat acclimation and mild dehydration. Brain Res 798: 127-139, 1998.

31. Simon E: Temperature regulation: the spinal cord as a site of extrahypothalamic thermoregulatory functions. Rev. Physiol. Biochem. Pharmacol. 71:1-76, 1974.

32. Kanosue K, Hosono Y, Zhang Y -H, and Chen X -M: Neuronal networks controlling thermoregulatory effectors. Progr. Brain Res. 115:49-62, 1998.

33. Kanosue K, Niwa K, Andrew P D, Yasuda H, Yanase M, Tanaka H, and Matsumura K: Lateral distribution of hypothalamic signals controlling thermoregulatory vasomotor activity and shivering in rats. Am J Physiol (Regul Integr Comp Physiol) 260: R486-R493, 1991.

34. Asami T., Hori T., Kiyohara T., and Nakashima T. Convergence of thermal signals on the reticulospinal neurons in the midbrain, pons and medulla oblongata. Brain Res. Bull. 20:581-586, 1988.

35. Zhang Y H, Hosono T, Yanase-Fujiwara M, Chen X -M, and Kanosue K: Effect of midbrain stimulations on thermoregulatory vasomotor responses in rats. J Physiol (Lond) 503: 177-186, 1997.

36. Nagashima K., Nakai S., Tanaka M., Kanosue K: Neuronal circuitries involved in thermoregulation. Auton. Neurosci. December 20;85(1-3):18-25, 2000.

37. Jessen C and Simon E: Spinal cord and hypothalamus as core sensors of temperature in the conscious dog. III. Identity of functions. Pflügers Arch 324: 217-226, 1971.

38. Puschmann S and Jessen C: Anterior and posterior hypothalamus: effects of independent temperature displacements on heat production in conscious goats. Pflügers Arch 373: 559-68, 1978,

39. Inomoto T, Mercer J B, and Simon E: Opposing effects of hypothalamic cooling on threshold and sensitivity of metabolic response to body cooling in rabbits. J Physic (Lond) 322: 139-150, 1982.

40. Andersson B and Larsson B: Influence of local temperature changes in the preoptic area and rostral hypothalamus on the regulation of food and water intake. Acta Physiol Scand 52: 75-89, 1961.

41. Baldwin B A and Cooper T R: Effects of warming or cooling the hypothalamus on food and water intake in sheep. J Physiol (Lond.) 332: 83P-84P, 1982.

42. Boulant J A: Cellular mechanisms of temperature sensitivity in hypothalamic neurons. Prog. Brain Res., 115, 3-8, 1998.

43. Boulant J A: Role of the preoptic-anterior hypothalamus in thermoregulation and fever. Clin. Infect. Dis. October;31 Suppl 5:S157-61, 2000.

44. Boulant J A: Hypothalamic neurons. Mechanisms of sensitivity to temperature. Ann. N. Y. Acad. Sci, 856, 108-115, 1998.

45. Boulant J A and Silva N L: Neuronal sensitivities in preoptic tissue slices: Interactions among homeostatic systems. Brain Res. Bull. 20 871-878, 1988.

46. Pierau Fr -K, Sann H, Yakimova K S, and Haug P: Plasticity of hypothalamic temperature-sensitive neurons. Progr Brain Res 115: 63-84, 1998.

47. Simon E, Rautenberg W, Thauer R, and Iriki M: Auslösung thermoregulatorischer Reaktionen durch lokale Kühlung im Vertebralkanal. Naturwissenschaften 50: 337, 1963.

48. Simon E, Rautenberg W, and Jessen C: Initiation of shivering in unanesthetized dogs by local cooling within the vertebral canal. Experientia 21:477, 1965.

49. Simon E, Klussman F W, Rautenberg W, and Kosaka M: Kältezittern bei narkotisierten spinalen Hunden. Pflügers Arch 291: 187-204, 1966.

50. Kosaka M, Simon E, and Thauer R: Shivering in intact and spinal rabbits during spinal cord cooling. Experientia.(Basel) 23: 385-387, 1967.

51. Kosaka M and Simon E: Der zentralnervöse, spinale Mechanismus des Kälterzitterns. Pflügers Arch 305: 357-373, 1968.

52. Inomoto T, Mercer J B, and Simon E: Interaction between hypothalamic and extrahypothalamic body temperatures in the control of panting in rabbits. Pflügers Arch 398: 142-146, 1983.

53. Silva N L and Boulant J A: Effects of osmotic pressure, glucose and temperature on neurons in preoptic tissue slices. Am J Physiol (Regul Integr Comp Physiol) 247: R335-R345, 1984.

54. Boulant J A and Silva N L: Interactions of reproductive steroids, osmotic pressure, and glucose on thermosensitive neurons in preoptic tissue slices. Can J Physiol Pharmacol 65: 1267-1272, 1987.

55. Hori T, Kiyohara T, Nakasima T, Shibata M, and Koga H: Multimodal responses of preoptic and anterior hypothalamic neurons to thermal and nonthermal homeostatic parameters. Can J Physiol Pharmacol 65: 1290-1298, 1987.

56. Travis K A and Johnson A K: In-vitro sensitivity of median preoptic neurons to angiotensin II, osmotic pressure and temperature. Am J Physiol (Regul Integr Comp Physiol) 264:R1200-R1205, 1993.

57. Travis K A and Boulant J A: In vitro osmosensitive hypothalamic neurons from hypertensive and normotensive rats. Am J Physiol (Regul Integr Comp Physiol) 258: R175-R181, 1990.

58. Jessen C: Two-dimensional determination of thermosensitive sites within the goat's hypothalamus. J Appl Physiol 40: 514-520, 1976.

59. Simon E, Pierau Fr -K, and Taylor D C M. Central and peripheral thermal control of effectors in homeothermic temperature regulation. Physiol. Rev. 66: 235-300, 1986.

60. Morimoto A, Murakami N, Nakamonr T, Watanabe T: Ventromedial hypothalamus is highly sensitive to prostaglandin E2 for producing fever in rabbits. J Physiol (Lond) 397:259-268, 1988.

61. Boulant J A: Hypothalamic neurons regulating body temperature. In: Handbook of Physology, Section 4, Environmental Physiology, Vol. 1, ed. Fregly M J and Blatteis C M, Am Physiol Soc, Oxford Univ Press, New York, pp 105-126, 1996.

62. Hori T and Katafuchi T: Cell biology and the functions of thermosensitive neurons in the brain. Progr Brain Res 115: 9-24,1998.

63. Simon E, Schmid H A, and Pehl U: Spinal neuronal thermosensitivity in vivo and in vitro in relation to hypothalamic neuronal thermosensitivity. Progr Brain Res 115: 25-48, 1998.

64. Griffin J D and Boulant J A: Physiological and morphological characteristics of hypothalamic temperature sensitive neurons. FASEB J 5: A1400, 1991.

65. Kanosue K, Nakayama T, Tanaka H, Yanase M, and Yasuda H: Modes of action of local hypothalamic and skin stimulations on salivary secretion in rats. J Physiol (Lond) 424: 459-471, 1990.

66. Kanosue K, Hosono T, and Yanase-Fujiwara M: Hypothalamic network for thermoregulatory vasomotor activity. Am J Physiol (Regul Integr Comp Physiol) 267: R283-R288,1994.

67. Chen X -M, Hosono T, Yoda T, Fukuda Y, and Kanouse K: Efferent projection from the preoptic area for the control of non-shivering thermogenesis in rats. J. Physiol. (Lond) 512:883-892, 1998.

68. Hare A S, Clarke G, and Tolchard S: Bacterial lipopolysaccharide-induced changes in Fos protein expression in the rat brain: correlation with thermoregulatory changes and plasma corticosterone. J Neuroendocrinol 7: 791-799, 1995.

69. Scammell T E, Price K J, and Sagar S M: Hyperthermia induces c-fos expression in the preoptic area. Brain Res 618: 303-307, 1993.

70. Gordon C J and Heath J E: Integration and central processing in temperature regulation. Annu. Rev. Physiol.;48:595-612, 1986.

71. Hori T, Nakashima T, Koga H, Kiyohara T, and Inoue T: Convergence of thermal, osmotic, and cardiovascular signals on preoptic and anterior hypothalamic neurons in the rat. Brain Res. Bull. 20:879-885, 1988.

72. Ninomiya I and Fujita S: Reflex effects of thermal stimulation on sympathetic nerve activity to skin and kidney. Am. J. Physiol. February;230(2):271-8, 1976.

73. Adair E R: Skin, preoptic, and core temperatures influence behavioral thermoregulation. J. Appl. Physiol. 42: 559-564, 1977.

74. Cheng C, Matsukawa T, Sessler D I, Ozaki M, Kurz A, Merrifield B. Lin H, and Olofsson P: Increasing mean skin temperature linearly reduces the core-temperature thresholds for vasoconstriction and shivering in humans. Anesthesiology 82: 1160-1168, 1995.

75. Crawshaw L, Nadel E, Stolwijk J, and Stamford B: Effect of local cooling on sweating rate and cold sensation. Pflügers Arch. 354: 19-27, 1975.

76. Jessen C: Independent clamps of peripheral and central temperatures and their effects of heat production in the goat. J. Physiol. (Lond.) 311: 11-22, 1981.

77. Keatinge W R, Mason A C, Millard C E, and Newstead C G: Effects of fluctuating skin temperature on thermoregulatory responses in man. J. Physiol. (Lond.) 378: 241-252, 1986.

78. Nadel E R, Horvath S M, Dawson C A, and Tucker A: Sensitivity to central and peripheral thermal stimulation in man. J. Appl. Physiol. 29: 603-609, 1970.

79. Sakurada S, Shido O, Fujikake K, and Nagasaka T: Relationship between body core and peripheral temperatures at the onset of thermoregulatory responses in rats. Jpn. J. Physiol. 43: 659-667, 1993.

80. Satinoff E: Behavioral thermoregulation in response to local cooling of the rat brain. Am. J. Physiol. 206:1389-1394, 1964.

81. Savage M V and Brengelmann G L: Control of skin blood flow in the neutral zone of human body temperature regulation. J. Appl. Physiol. 80: 1249-1257, 1996.

82. Wyss C R, Brengelmann G L, Johnson J M, Rowell L B, and Niederberger M: Control of skin blood flow, sweating, and heart rate: role of skin vs. core temperature. J. Appl. Physiol. 36: 726-733, 1974.

83. Cooper K E: The neurobiology of fever: thoughts on recent developments. Annu Rev Neurosci 10: 297-324, 1987.

84. Blatteis C M: Role of the OVLT in the febrile response to circulating pyrogens. Progr Brain Res 91: 409-412, 1992.

85. Zeisberger E and Merler G: The role of OVLT in fever and antipyresis. Progr Brain Res 91: 403-408, 1992.

86. Hashimoto M, Ueno T, and Iriki M: What role does the organum vasculosum laminae terminalis play in fever in rabbits. Pflügers Arch 429: 50-57, 1994.

87. Blatteis C M and Sehic E: Fever-how many circulating pyrogens signal the brain. News Physiol Sci 12: 1-9, 1997.

88. Walter J S, Meyers P, and Krueger J M: Microinjection of interleukin-1 into brain, separation of sleep and fever responses. Physiol Behav 45: 168-176, 1989.

89. Olorundare O E and Rudy T A: Examination of the subdiencephalic rat brain for sites mediating prostaglandin E1-induced pyrexia. Pharmacol Biochem Behav 25: 347-352,1986.

90. Kullmann R, Schönung W, and Simon E: Antagonistic changes of blood flow and sympathetic activity in different vascular beds following central thermal stimulation. I. Blood flow in skin, muscle and intestine during spinal cord heating and cooling in anesthetized dogs.

91. Walther O -E, Iriki M, and Simon E: Antagonistic changes in blood flow and sympathetic vascular beds following central thermal stimulation II. Cutaneous and visceral sympathetic activity during spinal cord heating and cooling in anesthetized rabbits and cats. Pflügers Arch 324:217-226, 1971.

92. Göbel D, Martin H, and Simon E: Primary cardiac responses to stimulation of hypothalamic and spinal cord temperature sensors evaluated in anesthetized paralyzed dogs J Therm Biol 2: 41-47, 1977.

93. Bell C, Jänig W, Kümmel H, and Xu H: Differentiation of vasodilator and sudomotor responses in the cat paw pad to preganglionic sympathetic stimulation. J Physiol (Lond) 364: 93-104, 1985.

94. Schönung W, Wagner H, and Simon E: Neurogenic vasodilatatory component in the thermoregulatory skin blood flow response of the dog. Naunyn-Schmiedebergs Arch Pharmacol 273: 230-241,1972.

95. Gregor M, Jänig W, and Riedel W: Response pattern of cutaneous postganglionic neurons to the hindlimb on spinal cord heating and cooling in the cat. Pflügers Arch 363: 135-140, 1976.

96. Lundberg J M, Änggad A, Fahrenkrug J, Hökfelt T, and Mutti V: Vasoactive intestinal polypeptide in cholinergic neurons of exocrine glands: functional significance of coexisting transmitters for vasodilation and secretion. Proc Natl Acad Sci USA 77: 1651-1655,1980.

97. Lundberg J, Norgren L, Rosen I, Steen S, Thörne J, and Wallin G: Direct evidence of active sympathetic vasodilatation in the skin of the human foot. J Physiol (Lond) 417: 241-255, 1989.

98. Sugenoya J, Ogawa T, Imai K, Ohnishi N, and Natsume K: Cutaneous vasodilatation responses synchronize with sweat expulsions. Eur J Appl Occupat Physiol 71: 33-40, 1995.

99. Mathai M L, Hjelmqvist H, Keil R, and Gerstberger R: Nitric oxide increases cutaneous and respiratory heat dissipation in conscious rabbits. Am J Physiol (Regul Integr Comp Physiol) 272: R1691-R1697, 1997.

100. Eriksson S, Hjelmqvist H, Keil R, and Gerstberger R: Central application of nitric oxide donor activates heat defense in the rabbit. Brain Res 774: 269-273, 1997.

101. Schmid H A, Riedel W, and Simon E: Role of nitric oxide in temperature regulation. Progr Brain Res 115: 87-110, 1998.

102. Pehl U, Schmid H A, and Simon E: Lamina-specific effects of nitric oxide in temperature sensitive neurons in spinal cord slices. In: Thermal Balance in Health and Disease, Advances in Pharmacological Sciences, ed. Zeisberger E, Schönbaum E, and Lomax P, Birkhäuser, Basel, pp 45-51, 1994.

103. Farrell D M and Bishop V S: Permissive role for nitric oxide in active thermoregulatory vasodilatation in rabbit ear. Am J Physiol (Heart Circ Physiol) 269: H1613-H1618, 1995.

104. Krönert H and Pleschka K: Lingual blood flow and its hypothalamic control in the dog during panting. Pflügers Arch 367: 25-31, 1976.

105. Pleschka K, Kühn P, and Nagai M: Differential vasomotor adjustments in the evaporative tissues of the tongue and nose in the dog under heat load. Pflügers Arch 382: 255-262, 1979.

106. Nagai M and Pleschka K: Brain stem sites mediating adrenergic and non-adrenergic vasodilatation in the dog's nose and tongue. J Auton Nerv Syst 4: 365-379, 1981.

107. Pleschka K: Control of tongue blood flow in regulation of heat loss in mammals. Rev Physiol Biochem Pharmacol 100: 75-120, 1984.

108. Lynn B, Schütterle S, and Pierau Fr -K: The vasodilator component of neurogenic inflammation is caused by a special subclass of heat-sensitive nociceptors in the skin of the pig. J Physiol (Lond) 494: 587-593, 1996.

109. Ikeda T and Pleschka K: Functional evidence of vasomotor control by tachykinins in the heat dissipating tissues of the canine nose and face. In: Thermal Balance in Health and Disease, Advances in Pharmacological Sciences, ed. Zeisberger E, Schönbaum E, and Lomax P, Birkhäuser, Basel, pp 209-214, 1994.

110. Pleschka K, Hashimoto M, Sommerland U, and Lürkens I: Distribution of facial and nasal blood flow in the cold loaded dog. J Therm Biol 12: 113-117, 1987.

111. Pleschka K, Sugahara M, Hashimoto M, Sommerland U, Lürkens I, and Ernst C: Local circulatory control in thermal stress. In: Comparative Physiology of Environmental Adaptations, ed. Dejours P, Krager, Basel, Vol. 2, pp 107-122, 1987.

112. Hales J R and Iriki M: Integrated changes in regional circulatory activity evoked by spinal cord and peripheral thermoreceptor stimulation. Brain Res. April 11;87(2-3):267-79, 1975.

113. Hori T, Nakashima T, Take S, Kaizuka Y, Mori T, and Katafuchi T: Immune cytokines and regulation of body temperature, food intake and cellular immunity. Brain Res Bull 27: 309-314, 1991.

114. Nijima A, Hori T, Aou S, and Oomura Y: The effects of interleukin 1beta on the activity of adrenal, splenic and renal sympathetic nerves in the rat. J Auton Nerv syst 36: 183-192, 1991.

115. Katafuchi T, Take S, and Hori T: Roles of sympathetic nervous system in the suppresion of cytotoxicity of splenic natural killer cells in the rat. J Physiol (Lond) 465: 343-357, 1993.

116. Take S, Mori T, Katafiuchi T, and Hori T: Central interferon-alfa inhibits natural killer cytotoxicity through sympathetic innervation. Am J Physiol (Regul Integr Comp Physiol) 265: R453-R459, 1993.

117. Ando T, Ichijo T, Katafuchi T, and Hori T: Intracerebroventricular injection of prostaglandin E-2 increases splenic sympathetic nerve activity in rats. Am J Physiol (Regul Integr Comp Physiol) 269: R662-R668, 1995.

118. Hori T, Katafchi T, Take S, Shimizu N, and Nijima A: The autonomic nervous system as a communication channel between the brain and the immune system. Neuroimmunomodulation 2: 203-215, 1995.

119. MacNeil A, Jansen A H, Greenberg H, and Nance D M: Activation and selectivity of splenic sympathetic nerve electrical activity response to bacterial endotoxin. Am J Physiol (Regul Integr Comp Physiol) 270: R264-R270, 1996.

120. Madden K S and Felten D L: Experimental basis for neural-immuno interactions. Physiol Rev 75:77-106, 1995.

121. Iriki M: Fever and fever syndrome-current problems. Jpn J Physiol 38: 233-250, 1988.

122. Riedel W, Kozawa E, and Iriki M: Renal and cutaneous vasomotor and respiratory rate adjustments to peripheral cold and warm stimuli and to bacterial endotoxin in conscious rabbits. J Auton Nerv Syst 5: 177-194, 1982.

123. Riedel W: Effects of propylthiouracil and of bacterial endotoxin on thyroid hormones, respiratory rate, cutaneous and renal blood flow in rabbits. Pflügers Arch 399: 11-17, 1983.

124. Riedel W: Mechanics of fever. J Basic Clin Physiol Pharmacol 1: 291-322, 1990.

125. Keil R, Riedel W, and Simon E: Hormonal secretion patterns but not autonomic effector responses elicited by hypothalamic heating and cooling are altered in febrile rabbits. J Auton Nerv Syst 57: 193-201, 1996.

126. Iriki M and Saigusa T: Regional differentiation of sympathetic efterents during fever. Progr Brain Res 115: 477-497, 1998.

127. Shimizu N, Kaizuka Y, Hori T, and Nakane H: Immobilization increases norepinephrine release and reduces NK cytotoxicity in spleen of conscious rats. Am J Physiol (Regul Integr Comp Physiol) 271: R537-R544, 1996.

128. Lacroix S, Vallieres L, and Rivest S: C-fos mRNA pattern and corticotropin releasing factor neuronal activity throughout the brain of rats injected centrally with a prostaglandin of E2 type. J Neuroimmunol 70: 163-179, 1996.

129. Scammell T E, Elmquivst J K, Griffin J D, and Saper C P: Ventromedial preoptic prostaglandin E2 activates fever-producing autonomic pathways. J Neurosci 16: 6246-6254, 1996.

130. Rolfe D F S and Brown G C: Cellular energy utilization and molecular origin of standard metabolic rate in mammals. Physiol Rev 77: 731-758, 1997.

131. Lange Andersen K, Hammel H T, Hildes G A, and Wilson O: A field study of physiological adjustment to increased muscular activity with and without cold exposure. Acta Univ Lund, Sect II, Part IV: 16-28, 1966.

132. Hammel H T, Simon E, Stromme S, and Lange Andersen K: Thermo and metabolic responses during a night of moderate cold exposure. Acta Univ Lund, Sect II, Part IV: 1-14, 1966.

133. Geiser F: Reduction of metabolism during hibernation and daily torpor in mammals and birds, temperature effect or physiological inhibition. J Comp Physiol B 158: 25-38, 1988.

134. Rothwell N J and Stock M J: Similarities between cold and diet-induced thermogenesis in the rat. Can J Physiol Pharmacol 57: 842-848, 1980.

135. Zhang Y. Proenca R, Maffei M, Barone M, Leopold L, and Friedman G M: Positional cloning of the mouse obese gene and its human homologue. Nature 372: 425-432, 1994.

136. Kershaw E E, Chua S C Jr, Williams J A, Murphy E M, and Leibel R: Molecular mapping of SSRs for Pgm 1 and C8b in the vicinity of the rat fatty locus. Genomics 27: 199-154,1995.

137. Chua S C Jr, Chung W K, Wu-Peng S, Zhang Y, Liu S -M, Tartaglia L, and Leibel R: Phenotypes of mouse diabetes and rat fatty due to mutations in the ob (leptin) receptor. Science 269: 546-549, 1996.

138. Kaul R, Schmidt I, and Carlisle H: Maturation of thermoregulation in the Zucker rats. Int J Obes 9: 401-409, 1985.

139. Planche E, Joliff M, de Gasquet P, and Leliepvre X: Evidence of a defect in energy expenditure in 7-day old Zucker rat (fa/fa). Am J Physiol (Endocrinal Metab) 245: E107-E113, 1983.

140. Schmidt I, Kaul R, and Carlisle H J: Body temperature of huddling new born Zucker rats. Pflügers Arch 401: 418-420, 1984.

141. Markewicz B. Kuhmichel G, and Schmidt I: Onset of excess fat deposition in Zucker ats with and without decreased thermogenesis Am J Physiol (Endocrinal Metab) 265: E478-486, 1993.

142. Kaul R, Heldmaier G, and Schmidt I: Defective thermoregulory thermogenesis does not cause onset of obesity in Zucker rats. Am J Physiol (Endocrinal Metab) 259: Evidence for decresed GDP binding to brown-adipose-tissue mitochondria of obese Zucker (fa/fa) rats in the very first days of life. Biochem J 221:241-245, 1984,

143. Körtner G, Petrova O, Vogt S, and Schmidt I: Sympathetically and nonsympathetically mediated onset of excess fat deposition in Zucker rats, Am J Physiol (Endocrinal Metab) 267: E947-953, 1994.

144. Wang T, Hartzell D L, Flatt W P, Marti R J, and Baile C A: Responses of lean and obese Zucker ras to centrally administred leptin. Physiol Behav 65: 333-341, 1998.

145. Stehling Döring Ertl J, Preibise G. Schmidt I: Leptin reduces juvenile fat stores by altering the circadian cycle of energy expenditure. Am J Physiol (Regul Integr Comp Physiol) 271: R1770-R1774, 1996.

146. Nusslein B and Schmidt I: Development of circadian of core temperature in juvenile rats Am J Physio (Regul Integr Comp Physiol) 259: R270-R276, 1990.

147. Imai-Matsumura K, Kaul R, and Schmidt I: Juvenle circadian core temperature rhythm in Wistar and lean (fa/−) Zucker rat pups. Physiol Behav 57: 135-139, 1995.

148. Spanswick D, Smith M A, Groppi V E, Logan S D, and Ashford M L J: Leptin inhibits hypothalamic neurons by activation of ATP sensitive potassium channels. Nature 390: 521-525, 1997

149. Vandenpol A N, Gao X B, Kilduff T S, and Belousov A B: Presynaptic and postsynaptic actions and moduliation of neuroendocrine neurons by a new hypothalamic peptide, hypocretin/orexin. J Neurosci 18: 7962-7971, 1998.

150. Horvath T L, Diano S, and van den Pol A N: Synaptic interaction between hypocretin and neuropeptide Y in the rodent and primate hypothalamus: a novel circuit implicated in metabolic and endocrine regulations. J. Neurosci 19: 1072-1087, 1999.

151. Rauch M, Riediger T, and Schmid H A: Antagonistic effects of orexin A and leptin on neurons in the arcuate nucleus of the rat. Pflügers Arch 437: R142, 1999.

152. Bornstein S R, Uhlmann K, Haidan A, Erhartrnstein M, and Scherbaum W A: Evidence for a novel peripheral action of leptin as a metabolic signal to the adrenal gland-leptin inhibits cortisol release directly. Diabetes 46: 1235-1238, 1997.

153. Cunningham M J, Clifton D K, and Steiner R A: Leptin's actions on the reproductive axis: perspectives and mechanisms. Biol Reproduct 60: 216-222, 1999.

154. Morimoto T: Thermoregulation and body fluids: Role of blood volume and central venous pressure. Jpn J Plysiol 40: 156-179, 1990.

155. Mack J W and Nadel E R: Body fluid balance during heat stress in humans. In: Handbook of Physiology, Section 4, Environmental physiology, ed. Fregly M J, and Blatteis C M, Am Physiol Soc, Oxford Univ Press, New York, Volume 1, pp 187-214, 1996.

156. Morimoto T, Itoh T, and Takamata A: Thermoregulation and body fluid in hot environments. Progr Brain Res 115: 499-508, 1998.

157. Doris P A and Baker M A: Intracranial osmoreceptors control evaporation in the heat-stressed cat. Brain Res 239: 644-648, 1982.

158. Baker M A, Doris P A, and Hawkins M J: Effect of dehydration and hyperosmolality on thermoregulatory water loss in the exercising dog. Am J Physiol (Regul Integr Com Physiol) 244: R516-R521, 1983.

159. Fortney S N, Nadel E R, Wenger C B, and Bove J R: Effect of blood volume on sweating rate and body fluid in exercising humans. J Appl Physiol 51: 1594-1600, 1981.

160. Fortney S M, Wenger C B, Bove J R, and Nadel E R: Effect of hyperosmolality on control of blood flow and sweating. J Appl Physiol 57: 1688-1695, 1984.

161. Baker M A: Effects of dehydration and rehydration on thermoregulatory sweating in goats. J Physiol (Lond) 417: 421-435, 1989.

162. Sawka M N, Latzka W A, Matott R P, and Montain S J: Hydration effects on temperature regulation. Int J Sports Med 19 (Suppl 2): S108-S110, 1998.

163. Yorimoto A, Nakai S, Yoshida T, and Morimoto T: Relationship between drinking behavior and body temperature during exercise in heat. Jpn J Phys Fitness Sports Med 44: 357-363, 1995.

164. Mack G W: Recovery after exercise in the heat: factors influencing fluid intake. Int J Sports Med 19 (Suppl 2): S139-S141, 1998.

165. Armstrong L E, Costill D L, and Fink W J: Changes in body water and electrolytes during heat acclimation, effects of dietary sodium. Aviat Space Environ Med 58:143-148, 1987.

166. Nielsen B, Hales J R S, Strange S, Christianson N J, Warberg J, and Saltin B: Human circulatory and thermoregulatory adaptations with heat acclimation and exercise in a hot dry environment. J Physiol (Lond) 460: 467-485, 1993.

167. Nielsen B, Strange S, Christianson N J, Warberg J, and Saltin B: Acute and adaptive responses in humans to exercise in a warm, humid environment. Pflügers Arch 434: 49-56, 1997.

168. Nielsen B: Heat acclimation: Mechanisms of adaptation to exercise in the heat. Int J Sports Med 19 (Suppl 2): S154-S156, 1998.

169. Zeisberger E, Roth J, and Simon E: Changes in water balance and release of arginine vasopressin during thermal adaptation in guinea-pigs. Pflügers Arch 412: 285-291, 1988.

170. Barney C C and Folkerts M M: Thermal dehydration-induced thirst in rats: role of body temperature. Am J Physiol (Regul Itegr Comp Physiol) 269: R557-R564, 1995.

171. Grace J E and Stevenson J A F: Themogenic drinking in the rat. Am J Physiol 220: 1009-1015, 1971.

172. Olsson K, Josater-Hermlin M, Hossaini-Hilai J, Hydbring E, and Dahlborn K: Heat stress causes excessive drinking in fed and food-deprived pregnant goats. Comp Biochem Physiol A110: 309-317, 1995.

173. Rautenberg W, May B, and Arabin G: Behavioral and autonomic temperature regulation in competiton with food intake and water balance of pigeons. Pflügers Arch 384: 253-260, 1980.

174. Brummermann M and Rautenberg W. Interaction of autonomic and behavioral thermoregulation in osmotically stressed pigeons. Physiol Zool 62: 1102-1106, 1989.

175. Honda K, Negoro H, Dyball R E J, Higuchi T, and Takano S: The osmoreceptor complex in the rat: evidence for interactions between the supraoptic and other diencephaic nuclei J Physiol (Lond) 431: 225-241, 1990.

176. Korf H -W, Simon-Oppermann C, and Simon E: Affrent connections of physiologically identified neuronal complexes in the paraverticular nucleus of conscious Pekin ducks involved in regulation of salt- and water-balance. Cell Tissue Res 226: 275-300, 1982.

177. Kanosue K, Schmid H, and Simon E: Differential osmoresponsiveness of periventricular neurons in duck hypothalamus Am J Physiol (Regul Integr Comp Physiol) 258: R973-R981, 1990.

178. Kanosue K, Gerstberger R, Simon-Oppermann C, and Simon E: Ionic responsiveness in third ventricular hypertonic stimulation of antidiuresis in ducks. Brain Res 569: 268-2274, 1992

179. Simon E: Thermoregulation as a Switchboard of Autonomic Nervous and Endocrine Control. Jpn J Physiol 49: 297-323, 1999.

180. Darian-Smith I and Johnson K O Thermal sensibility and thermoreceptors. J Invest Dermatol 69: 146-153, 1977.

181. Darian-SmithI: Thermal sensibility. In Handbook of Physiology, The Nervous System Vol III, American Physiological Society, Bethesda, Mass., pp. 879-913, 1984.

182. Danian-Smith I, Johnson K O, and Dykes R: “Cold” fiber population innervating pahmar and digital skin ofthe monkey: Responses to cooling pulses. J. Neurophysiol 36:325-346, 1973.

183. Spray D C: Cutaneous temperature receptors. Annu Rev Physiol 48: 625-638, 1986.

184. Gee M D, Lynn B, Basile S, Pierau F K, Cotsell B: The relationship between axonal spike shape and functional modality in cutaneous C-fibres in the pig and rat. Neuvoscience; May;90(2):509-18, 1999.

185. Dollé P, Izpisua-Belmonte J C, Falkenstein H, Renucci A, and Duboule D: Coordinate expression of the murine Hox-5 complex homeobox-containing genes during limb pattern formation. Nature 342, 767-772, 1989.

186. Duboule D: The function of Hox genes in the morphogenesis of the vertebrate limb. Ann. Genet.;36(1):24-9, 1993.

187. Duboule D: The vertebrate limb: a model system to study the Hox/HOM gene network during development and evolution. Bioessays June;14(6):375-84, 1992.

188. Fallon J F, López A, Ros M A, Savage M P, Olwin B B, and Simandl B K: FGF-2: apical ectodermal ridge growth signal for chick limb development. Science 264, 104-107, 1994.

189. Haack H, and Gruss P: The establishment of murine Hox-1 expression domains during patterning of the limb. Dev. Biol. 157, 410-422, 1993.

190. Hardy A, Richardson M K, Francis-West P H, Rodriguez C, Izpisua-Belmonte J C, Duprez D, and Wolpert L: Gene expression, polarising activity and skeletal patterning in reaggregated hind limb mesenchyme. Development 121, 4329-4337, 1995.

191. Koyama E, Noji S, Nohno T, Myokai F, Ono K, Nishijima K et al: Cooperative activation of Hoxd Homoeobox genes by factors from the polarizing region and the apical ridge in chick limb morphogenesis. Develop. Growth & Differ. 35, 189-198,1993.

192. Krumlauf R: Hox genes in vertebrate development. Cell 78, 191-201, 1994.

193. Laufer E et al: Expression of Radical fringe in limb bud ectoderm regulates apical ectodermal ridge formation. Nature 366, 373, 1997.

194. Loomis C A, Harris E, Michaud J. Wurst W, Hanks M, and Joyner W L: The mouse Engrailed-1 gene and ventral limb patterning. Nature 382, 360-363, 1996.

195. López-Martinez A, Chang D T, Chiang C, Porter J A, Ros M A, Simandl B K et al: Limb-patterning activity and restricted posterior localization of the amino-terminal product of Sonic hedgehog cleavage. Curr. Biol. 5: 791-796, 1995.

196. Michaud J L, Lapointe F, and Le Douarin N M: The dorsoventral polarity of the presumptive limb is determined by signals produced by the somites and by the lateral somatopleure. Development 124: 1453-1463, 1997.

197. Morgan B A, Tabin C: Hox genes and growth: early and late roles in limb bud morphogenesis. Dev. Suppl:181-6, 1994.

198. Niswander L, Tickle C, Vogel A, Booth I, and Martin G R: FGF-4 replaces the apical ectodermal ridge and directs outgrowth and patterning of the limb. Cell 75: 579-587, 1993.

199. Niswander L, Jeffrey S, Martin G R, and Tickle C: A positive feedback loop coordinates growth and patterning in the vertebrate limb. Nature 371, 609-612, 1994.

200. Parr B A and McMahon A P: Dorsalizing signal Wnt-7a required for normal polarity of D-V and A-P axes of mouse limb. Nature 374: 350-353, 1995.

201. Riddle R D, Ensini M, Nelson C, Tsuchida T, Jessel T M, and Tabin C: Induction of the LIM homeobox gene Lmx1 by WNT7a establishes dorsoventral pattern in the vertebrate limb. Cell 83: 631-640, 1995.

202. Riddle R D, Johnson R L, Laufer E, and Tabin C: Sonic hedgehog mediates the polarizing activity of the ZPA. Cell 75: 1401-1416, 1996.

203. Ros M A, Lyons G E, Mackem S, and Fallon J F: Recombinant limbs as a model to study homeobox gene regulation during limb development. Dev. Biol. 166: 59-72, 1994.

204. Ros M A et al: The limb field mesoderm determines initial limb bud anteroposterior asymmetry and budding independent of sonic hedgehog or apical ectodermal gene expressions. Development 122: 2319-2330, 1996.

205. Tabin C: Retinoids, Homeoboxes, and Growth factors: toward molecular models for limb development. Cell 66: 199-217, 1991.

206. Tickle C and Eichele G: Vertebrate limb development. Ann. Rev. Cell Biol. 10: 121-152, 1994.

207. Vogel A, Rodriguez C, Wamken W, and lzpisua Belmonte J C: Dorsal cell fate specified by chick Lmx1 during vertebrate limb development. Nature 378: 716-720, 1995.

208. Wahba G M, Hostikka S L, Carpenter E M: The Paralogous Hox Genes Hoxa10 and Hoxd10 Interact to Pattern the Mouse Hindlimb Peripheral Nervous System and Skeleton. Dev. Biol. March 1;231(1):87-102, 2001.

209. Yang Y, and Niswander L: Interaction between the signalling molecules WNT7a and SHH during vertebrate limb development: dorsal signals regulate anteroposterior patterning. Cell 80: 939-937, 1995.

210. Yokouchi Y, Sasaki H. and Kuroiwa A: Homeobox gene expression correlated with the bifurcation process of limb cartilage development. Nature 353: 443-445, 1991.

211. Zakany J and Doubule D: Hox genes in digit development and evolution. Cell Tissue Res. April;296(1):27-31, 1999.

212. Reid G and Flonta M L: Physiology. Cold current in thermoreceptive neurons. Nature October 4;413(6855):480, 2001.

213. Cesare P, Moriondo A, Vellani V and McNaughton P A: Ion channels gated by heat. Proc. Natl Acad. Sci. USA, 96: 7658-7663, 1999.

214. Braun W, Eckhardt B, Braun H A, Huber M: Phase-space structure of a thermoreceptor. Phys Rev E Stat Phys Plasmas Fluids Relat Interdiscip Topics November;62(5 Pt A):6352-60, 2000.

215. Nagy I and Rang H P: Similarities and differences between the responses of rat sensory neurons to noxious heat and capsaicin. J. Neurosci 19: 10647-10655, 1999.

216. Maingret F, Lauritzen I, Patel A J, Heurteaux C, Reyes R, Lesage F, Lazdunski M, Honore E. TREK-I is a heat-activated background K(+) channel. EMBO J June 1 ;19(11):2483-91.n et al 1980, 2000.

217. Tominaga M, Caterina M J, Malmberg A B, Rosen T A, Gilbert H, Skinner K, Raumann B E, Basbaum A I and Julius D: The cloned capsaicin receptor integrates multiple pain-producing stimuli. Neuron, 21: 531-543, 1998.

218. Nagy I and Rang H: Noxious heat activates all capsaicin-sensitive and also a sub-population of capsaicin-insensitive dorsal root ganglion neurons. Neuroscience, 88: 995-997, 1999.

219. Schaffer K, and Braun H A: Modulation of cutaneous cold receptor function by electrolytes, hormones and thermal adaptation: Physiol Res 41: 71-75, 1992.

220. Ivanov K P: Physiological problems and functional mechanisms of the thermoregulation system. Ann N Y Acad Sci 813:32-8, 1997.

221. Ivanov K P, Dymnikova L P et al: [The “thermoresponsive” neurons of the thermoregulation center and their functional characteristics]. Biull Eksp Biol Med 116(7): 11-3, 1993.

222. Jessen C: Thermal afferents in the control of body temperature. Pharmacol Ther 28(1): 107-34, 1985.

223. Simon E: The enigma of deep-body thermosensory specificity. Int J Biometeorol 44(3): 105-20, 2000.

224. Brock J A, Pianova S, Belmonte C: Differences between nerve terminal impulses of polymodal nociceptors and cold sensory receptors of the guinea-pig cornea. J Physiol June 1 ;533(Pt 2):493-501, 2001.

### BRIEF SUMMARY OF THE INVENTION

In accordance with the present invention, a method for enhancing and regularizing autonomic nervous and endocrine function is disclosed whereby specific discrete skin areas are stimulated with copper and aluminum plates. The discriminate stimulation of such specific discrete skin areas with copper and aluminum plates enhances, in turn, specific autonomic nervous and endocrine functions.

The method consists of applying one of the following procedures:

1. Externally placing two cold (colder than skin temperature) copper plates, wrapped and soaked in a solution, in strategic specific discrete skin areas during some hours of the day and/or night.

2. Externally placing one copper plate and one aluminum plate (4×1 cm), wrapped and soaked in a solution, in strategic specific discrete skin areas during some hours of the day and/or night.

In this way specific regions of the autonomic nervous and endocrine systems are stimulated and their functions are enhanced and regularized.

One of the most intended applications of the is the regularization of menstrual cycles and the enhancement of fertility. A different specific discrete skin area is to be stimulated continuously during each specific phase of the menstrual cycle.

Many other applications of the invention exist. Some examples are: the synthesis of particular hormones can be enhanced; the performance of the immune system can be incremented; salt and fluid balance can be modified; hunger can be enhanced; and specific metabolic pathways can be influenced.

The method is very safe, drug free, highly efficient, and very easy to apply.

### BRIEF DESCRIPTION OF THE DRAWINGS

There is a drawing for each of the twenty-eight specific discrete skin areas to be stimulated with copper and aluminum plates. Seven different positions have been designated for the forearm, seven for the hand, seven for the lower leg and seven for the foot. The specific discrete skin areas to be stimulated are filled in black.

### DETAILED DESCRIPTION OF THE INVENTION

I. Description of the Theoretical Basis of the Invention

II. Description of a Clinical Case

III. Brief Description of what we Claim to be Novel

1. Description of the Theoretical Basis of the Invention

Temperature regulation involves integration of autonomic, endocrine, and skeletomotor responses. Neuronal circuits involved in thermoregulatory control are complex. Effector areas for specific thermoregulatory responses are located throughout the brain stem and spinal cord. Thermosensitive neurons in and near the anterior/preoptic hypothalamus respond to subtle changes in hypothalamic or core temperature. Such neurons, which also receive input from visceral, skin, and spinal thermoreceptors, compare and integrate central and peripheral information, acting as a coordinating center that strongly influences each of the lower effector areas, eliciting in this way the optimal thermoregulatory responses for both internal and environmental thermal conditions. Thermal receptor afferents from different tissues affect thermoregulation in different ways. In addition, other humoral or endogenous factors, such as pyrogens, are integrated.

Sympathetic control of cardiovascular and immune functions, hormonal control of energy balance, and neurohormonal control of salt and fluid balance have been reviewed and analyzed in as much as they are challenged by competing demands of thermoregulatory requirements. These interactions illustrate the close relationship of non-thermoregulatory control systems with thermoregulatory activation and vice versa, and constitute a very useful analytical and experimental tool to elucidate the cytoarchitecture of neuronal circuits by which specific endocrine and autonomic regulatory activities are controlled.

Skin thermal sensory systems consist of separate receptors of warmth and cold, which are arranged in separate spots on the skin where thermal stimulation elicits the sensation of either warmth or cold. The threshold for eliciting a thermal sensation at these spots is considerably lower than in surrounding regions of the skin. Cold and warmth spots correspond to discrete zones of innervation by cold and warmth receptors whose pattern of distribution differs from one specific discrete skin area to another.

Specialized cold and warmth receptors are present in dorsal root ganglion sensory fibers as well as in the anterior/preoptic hypothalamus. Presumably, the afferent pathways related to discrete groups of cold or warmth sensitive spots on the skin reach specific points of the thermoregulatory circuitry, eliciting specific thermoregulatory responses.

The present invention is based on the assumption that the continuous and discriminate stimulation of specific discrete thermal receptors influences thermoregulation is specific ways.

The present invention takes advantage of such interactions by stimulating specific discrete skin areas directly integrated in thermoregulatory control systems, which in turn enhance specific endocrine and autonomic functions.

The precise location of each specific discrete skin area to be stimulated has been originally designated arbitrarily in the feet, hands, forearms, and lower parts of the legs. Seven different positions, or locations—two in the dorsal side, three in the ventral side, one in the anterior side, and one in the posterior side—have been assigned to each of these parts of the body. Each of these positions: forearm ventral anterior (fava), forearm ventral medial (favm), forearm ventral posterior (favp), forearm posterior (fap), forearm dorsal posterior (fadp), forearm dorsal anterior (fada), forearm anterior (faa), hand ventral anterior (hva), hand ventral medial (hvm), hand ventral posterior (hvp), hand posterior (hp), hand dorsal posterior (hdp), hand dorsal anterior (hda), hand anterior (ha), leg ventral anterior (lva), leg ventral medial (lvm), leg ventral posterior (lvp), leg posterior (lp), leg dorsal posterior (ldp), leg dorsal anterior (lda), leg anterior (la), foot ventral anterior (fva), foot ventral medial (fvm), foot ventral posterior (fvp), foot posterior (fp), foot dorsal posterior (fdp), foot dorsal anterior (fda), and foot anterior (fa) is represented in each of the twenty-eight drawings respectively. Efforts are being made to fine-tune these locations through experimental research and through the analysis of the pattern of expression of Hox gene complexes during limb formation in vertebrate embryos. A subset of members of the HoxD complex is expressed in a series of domains ordered along one limb axis, and a subset of members of the HoxA complex is expressed in series along a different axis.

During development, Hox genes help specify the differences between body segments. They code for DNA-binding proteins whose expression patterns help subdivide the body into developmental compartments. Subsequent processes generate a fine-grained pattern of cell differentiation inside each compartment, helping organize the creation of minutely specialized sets of cells, forming structures such as thermal sensitive complexes.

In accordance to the theoretical basis and performance of the present invention, it is proposed that the pattern of expression of Hox, and some other regulatory genes, expressed during developmental stages delimit specific discrete skin areas whose discriminate stimulation enhances specific autonomic and endocrine functions in response to thermoregulatory activation.

Many of the specific autonomic and endocrine responses that the proper stimulation of each specific discrete skin area elicits are still to be clarified through experimental research.

The mechanism by which thermal receptors are activated is not clearly understood in molecular terms. Although the molecular basis of heat sensitive neurons is beginning to be elucidated, knowledge about cold sensitive neurons is still lacking. It has been suggested that some skin receptors involved in thermal perception are polymodal.

The present invention stimulates receptors located in discrete skin areas using one of the following procedures:

1. Applying a direct cold stimulus to such specific discrete skin areas with cold copper plates.

2. Creating an electromagnetic flow using such specific discrete skin areas as terminals by placing a copper plate in the specific discrete skin area in one side of the body and an aluminum plate in the specific discrete skin area of the symmetrical side of the body.

The copper and aluminum plates have a potential difference of approximately 0.6 Volts. The plates are wrapped in a cotton material and soaked in a saline solution, which makes conduction to the skin efficient. Once the copper and aluminum plates—previously wrapped and soaked in the saline solution—are placed on the specific discrete skin area to be stimulated, the plates are covered with a plastic material in order to keep them humid and maintain conductance.

The present invention succeeds in enhancing and regularizing specific autonomic nervous and endocrine functions by discriminately applying the proper stimuli (cold or electromagnetic) to specific discrete skin areas.

### EXAMPLE

### Regularization of Menstrual Cycles and Entrancement of Fertility

Four different specific discrete skin areas are to be stimulated throughout the menstrual cycle. The electromagnetic stimulation of the first specific discrete skin area triggers autonomic and endocrine responses that enhance the functions of the menstruation days; the electromagnetic stimulation of the second specific discrete skin area triggers autonomic and endocrine responses that enhance the functions of the follicular phase of the cycle; the electromagnetic stimulation of the third specific discrete skin area triggers autonomic and endocrine responses that enhance the functions of ovulation; and the electromagnetic simulation of the fourth specific discrete skin triggers autonomic and endocrine responses that enhance the functions of the luteal phase of the cycle. Menstrual cycles will regularize and fertility will be enhanced if the four different specific discrete skin areas are stimulated during the proper days throughout the cycle.

II. Description of a Clinical Case

One clinical case is exposed herein in order to illustate the performance of the discriminate stimulation of specific discrete skin areas with copper and aluminum plates in the enhancement and regularization of specific autonomic nervous and endocrine functions. It is strongly suggested that specific autonomic nervous and endocrine functions can be enhanced and regularized by the discriminate stimulation of specific discrete skin areas with copper and aluminum plates.

A 33-year-old woman had unsuccessfully attempted to become pregnant for more than eight years. The woman had been previously treated systematically and unsuccessful during five years with several drugs and methods already approved and commonly used by Medical Doctors to enhance fertility.

One year after she had quit all the other treatments, our procedure was applied to her, consisting of the following steps:

1. Placing one copper plate (4×1 cm, wrapped in a cotton material, and soaked in a saline solution) on the skin surface in position favm (refer to the drawings) on the right side and placing one aluminum plate (4×1 cm, wrapped in a cotton material, and soaked in a saline solution) on the skin surface in position favm (refer to the drawings) on the left side. Each plate—previously cleaned with a knife, wrapped in a cotton material, soaked in a saline solution, and placed in the position described above—was covered with a plastic material and held in place with a bandage every night during the days of menstruation of the first cycle of treatment.

2. Placing one copper plate (4×1 cm, wrapped in a cotton material, and soaked in a saline solution) on the skin surface in position faa (refer to the drawings) on the right side and placing one aluminum plate (4×1 cm, wrapped in a cotton material, and soaked in a saline solution) on the skin surface in position faa (refer to the drawings) on the left side. Each plate—previously cleaned with a knife, wrapped in a cotton material, soaked in a saline solution, and placed in the position described above—was covered with a plastic material and held in place with a bandage every night for the rest of that first cycle of treatment.

3. Placing one copper plate (4×1 cm, wrapped in a cotton material, and soaked in a saline solution) on the skin surface in position favm (refer to the drawings) on the right side and placing one aluminum plate (4×1 cm, wrapped in a cotton material, and soaked in a saline solution) on the skin surface in position favm (refer to the drawings) on the left side. Each plate—previously cleaned with a knife, wrapped in a cotton material, soaked in a saline solution, and placed in the position described above—was covered with a plastic material and held in place with a bandage every night during the days of menstruation of the second cycle of treatment.

4. Placing one copper plate (4×1 cm, wrapped in a cotton material, and soaked in a saline solution) on the skin surface in position faa (refer to the drawings) on the right side and placing one aluminum plate (4×1 cm, wrapped in a cotton material, and soaked in a saline solution) on the skin surface in position faa (refer to the drawings) on the left side. Each plate—previously cleaned with a knife, wrapped in a cotton material, soaked in a saline solution, and placed in the position described above—was covered with a plastic material and held in place with a bandage every night during days four to nine of that second cycle of treatment.

5. Placing one copper plate (4×1 cm, wrapped in a cotton material, and soaked in a saline solution) on the skin surface in position fadp (refer to the drawings) on the right side and placing one aluminum plate (4×1 cm, wrapped in a cotton material, and soaked in a saline solution) on the skin surface in position fadp (refer to the drawings) on the left side. Each plate—previously cleaned with a knife, wrapped in a cotton material, soaked in a saline solution, and placed in the position described above—was covered with a plastic material and held in place with a bandage every night during days ten to sixteen of that second cycle of treatment.

6. Placing one copper plate (4×1 cm, wrapped in a cotton material, and soaked in a saline solution) on the skin surface in position fap (refer to the drawings) on the right side and placing one aluminum plate (4×1 cm, wrapped in a cotton material, and soaked in a saline solution) on the skin surface in position fap (refer to the drawings) on the left side. Each plate—previously cleaned with a knife, wrapped in a cotton material, soaked in a saline solution, and placed in the position described above—was covered with a plastic material and held in place with a bandage every night during the rest of that second cycle of treatment.

Results

The woman became pregnant during the second cycle of treatment.

Conclusion

Although a study with several women is necessary to assess the precise performance of this application of the invention, this case illustrates one of its most intended applications, and suggests that the treatment applied to the woman enhances fertility.

III. Brief Description of What we Claim to be Novel

A. The discovery that the continuous and discriminate stimulation of specific discrete skin receptors with copper and aluminum plates, previously wrapped in a cotton material and soaked in a saline solution, enhances specific autonomic nervous and endocrine functions.

B. The finding and designation of the specific discrete skin areas to be stimulated during each phase of the menstrual cycle in order to regularize the cycle and enhance fertility.

C. The finding and designation of the specific discrete skin area to be stimulated for other specific applications of the method, such as enhancing the performance of the immune system.

D. The proposition stating that the pattern of expression of Hox and some other regulatory genes expressed during developmental stages delimit specific discrete skin areas whose discriminate stimulation enhances specific autonomic and endocrine functions in response to thermoregulatory activation.

E. The proposition stating that some polymodal skin receptors can be activated creating an electromagnetic flow by placing a copper plate in a specific discrete skin area and an aluminum plate in another.

We claim:

1. A method of enhancing and regularizing autonomic nervous and endocrine function consisting of placing cold (colder than skin temperature) copper plates (4×1 cm, wrapped in a cotton material, and soaked in a saline solution) on the skin surface in any of the twenty eight positions described in the drawings.

2. A method of enhancing and regularizing autonomic nervous and endocrine function consisting of:
(a) placing one copper plate (4×1 cm, wrapped in a cotton material, and soaked in a saline solution) on the skin surface in any of the twenty eight positions described in the drawings, and
(b) placing one aluminum plate (4×1 cm, wrapped in a cotton material, and soaked in a saline solution) on the skin surface in any of the twenty eight positions described in the drawings.

3. A method of regularizing menstrual cycles and enhancing fertility in women consisting of the steps of:
(1a) placing one copper plate (4×1 cm, wrapped in a cotton material, and soaked in a saline solution) on the skin surface in position favm (refer to the drawings) on the right side and placing one aluminum plate (4×1 cm, wrapped in a cotton material, and soaked in a saline solution) on the skin surface in position favm (refer to the drawings) on the left side, every night during the days of menstruation.
(1b) placing one copper plate (4×1 cm, wrapped in a cotton material, and soaked in a saline solution) on the skin surface in position faa (refer to the drawings) on the right side and placing one aluminum plate (4×1 cm, wrapped in a cotton material, and soaked in a saline solution) on the skin surface in position faa (refer to the drawings) on the left side, every night for the rest of the cycle. {steps 1a and 1b are to be applied during the first cycle of treatment. Steps 2a, 2b, 2c, and 2d are to be applied during the second and following cycles of treatment}
(2a) placing one copper plate (4×1 cm wrapped in a cotton material and soaked in a saline solution) on the skin surface in position favm (refer to the drawings) on the right side and placing one aluminum plate (4×1 an, wrapped in a cotton material, and soaked in a saline solution) on the skin surface in position favm (refer to the drawings) on the left side, every night during the days of menstruation.
(2b) placing one copper plate (4×1 cm, wrapped in a cotton material, and soaked in a saline solution) on the skin surface in position faa (refer to the drawings) on the right side and placing one aluminum plate (4×1 cm, wrapped in a cotton material, and soaked in a saline solution) on the skin surface in position faa (refer to the drawings) on the left side, every night during the follicular phase of the cycle.
(2c) placing one copper plate (4×1 cm, wrapped in a cotton material, and soaked in a saline solution) on the skin surface in position fadp (refer to the drawings) on the right side and placing one aluminum plate (4×1 cm, wrapped in a cotton material, and soaked in a saline solution) on the skin sure in position fadp (refer to the drawings) on the left side, every night two days before, during, and two days after ovulation.
(2d) placing one copper plate (4×1 cm, wrapped in a cotton material, and soaked in a saline solution) on the skin surface in position fap (refer to the drawings) on the right side and placing one aluminum plate (4×1 cm, wrapped in a cotton material, and soaked in a saline solution) on the skin surface in position fap (refer to the drawings) on the left side, every night during the luteal phase of the cycle.

4. A method of enhancing the functions of the immune system consisting of
(a) placing one copper plate (4×1 cm, wrapped in a cotton material, and soaked in a saline solon) on the skin surface in position fava (refer to the drawings) in one side of the body, and
(b) placing one aluminum-plate (4×1 cm, wrapped in a cotton material, and soaked in a saline solution) on the skin surface in position fava (refer to the drawings) in the opposite, symmetrical side of the body.
